(19) United States
(12) Patent Application Publication
(10) Pub. No.: US 2018/0103859 A1
(43) Pub. Date: Apr. 19, 2018

(54) Systems, Devices, and/or Methods for Managing Patient Monitoring

(71) Applicant: Catalina F Provenzano, Annapolis, MD (US)

(72) Inventor: Catalina F Provenzano, Annapolis, MD (US)

(21) Appl. No.: 15/664,987

(22) Filed: Jul. 31, 2017

Related U.S. Application Data
(60) Provisional application No. 62/369,075, filed on Jul. 30, 2016.

(51) Int. Cl. A61B 5/024; A61B 5/0205; A61B 5/00

(52) U.S. Cl. CPC A61B 5/02438; A61B 5/0205; A61B 5/1112; A61B 5/0024; A61B 5/681

(57) ABSTRACT

Certain exemplary embodiments can provide an bracelet wearable by a user on a wrist of the user. The bracelet comprises a processor communicatively coupled to an information device of a medical care facility, wherein the information device is constructed to receive and store vital sign information for a plurality of patients of the medical care facility. The processor is constructed to request and receive vital sign information from the information device.

### CROSS-REFERENCES TO RELATED APPLICATIONS

This application claims priority to, and incorporates by reference herein in its entirety, pending U.S. Provisional Patent Application Ser. No. 62/369,075 (Attorney Docket No. 1158-01), filed Jul. 30, 2016.

### BRIEF DESCRIPTION OF THE DRAWINGS

A wide variety of potential practical and useful embodiments will be more readily understood through the following detailed description of certain exemplary embodiments, with reference to the accompanying exemplary drawings in which:

FIG. 1 shows a device in the form of a smartwatch;

FIG. 2 shows a device in the form of a bracelet;

FIG. 3 is a block diagram of an exemplary embodiment of an information device 3000; and

FIG. 4 is a flowchart of an exemplary embodiment of a method 4000.

### DETAILED DESCRIPTION

Certain exemplary embodiments can provide an bracelet wearable by a user on a wrist of the user. The bracelet comprises a processor communicatively coupled to an information device of a medical care facility, wherein the information device is constructed to receive and store vital sign information for a plurality of patients of the medical care facility. The processor is constructed to request and receive vital sign information from the information device.

Hospitals and/or other medical care facilities can monitor patient vital signs on a substantially continuous basis. Vital signs can be used to determine the deterioration of a patient's condition. An early warning sign system can measure temperature, blood pressure, pulse, and respiratory rate, which can be used to reduce a count of deaths in hospitals due to cardiac arrest. However, even though hospitals have technology to substantially continuously measure vital signs, they might lack the ability for nurses to substantially continuously monitor a patient's vital signs when the nurses are not in the patient's room. In certain facilities, nurses could be assisted in recognizing and more quickly responding to possible problems in a patient's vital signs.

Nurses have many jobs to do and only approximately 37% of their time is spent with their patients. Yet nurses could improve their services by staying in contact with their patients' statuses during times when no one is present in a room of a particular patient. Many hospitals have TV screens and computers that render vital signs, however nurses are always on the move, and are not always able to watch the screens. So a wearable device could be beneficial for nurses to be able to check patient vital signs on a substantially continuous basis. Furthermore, nurses might not feel confident in calling the rapid response team when they believe a particular patient has a problem. A major reason for this is that they are nervous about seeming inadequate, or scared that they are calling doctors impulsively and without good reason. If there were to be a device developed that told nurses when vital signs indicate a serious problem, the fear would be diminished as the pressure of decision making would be placed on a computer. Certain exemplary embodiments provide a compact bracelet that indicates changes of vitals that might be dropping or raising to dangerous levels. The purpose of the device is to improve treatment of conditions such as cardiopulmonary arrests or other such maladies that can cause sudden death by notifying a staff person such as a nurse of possibly dangerous changes in the patient's vitals that they may not be able to see. By decreasing the time of response, the patient can have a better chance of survival. Certain exemplary embodiments can be helpful to nurses with multiple patients and can allow them to treat patients while simultaneously monitoring other patients without having to carry around a larger device.

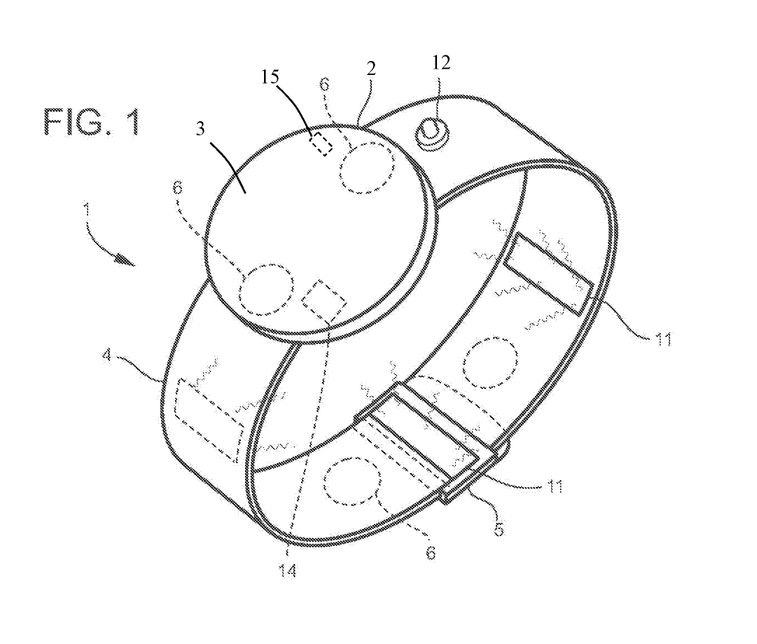

FIG. 1 shows a device in the form of a smartwatch. The smartwatch can be communicatively coupled to a network of a medical care facility (e.g., a hospital, clinic, and/or assisted living facility, etc. Each of a plurality of patients at the medical care facility can be monitored as to vital signs. For example, monitored vital signs can comprise body temperature, pulse rate, respiration rate (rate of breathing), blood pressure, pulse oximetry, blood glucose level, and/or end-tidal carbon dioxide, etc. Each monitored vital sign has a predetermined acceptable range and/or rate of change, which is provided to the smartwatch and/or an information device communicatively coupled to the smartwatch.

The smartwatch can be used by medical professionals such as a physicians, physician assistants, nurses, advanced practice registered nurses, operating department practitioners, emergency medical technicians, and/or paramedics, etc. For example, a medical professional (e.g., a nurse) can be assigned to a predetermined plurality of patients. The smartwatch can be communicatively coupled to vital sign monitors for each of the predetermined plurality of patients. Via the smartwatch, the medical professional can select a particular patient of the predetermined plurality of patients and the vital signs of the particular patient will be rendered on the smartwatch.

The smartwatch is also capable of providing a sensory alert to the medical practitioner if one of the plurality of patients exhibits a change in vital signs that warrants intervention. For example, a predetermined change in any of the set of vital signs can invoke the sensory alert. In addition, if one or more of the vital signs of one of the plurality of patients exceeds a predetermined threshold level, the sensory alert is triggered. Responsive to the sensory alert, the medical practitioner can take corrective action to avoid death or other serious consequences that might occur as a consequence of the cause of the sensory alert.

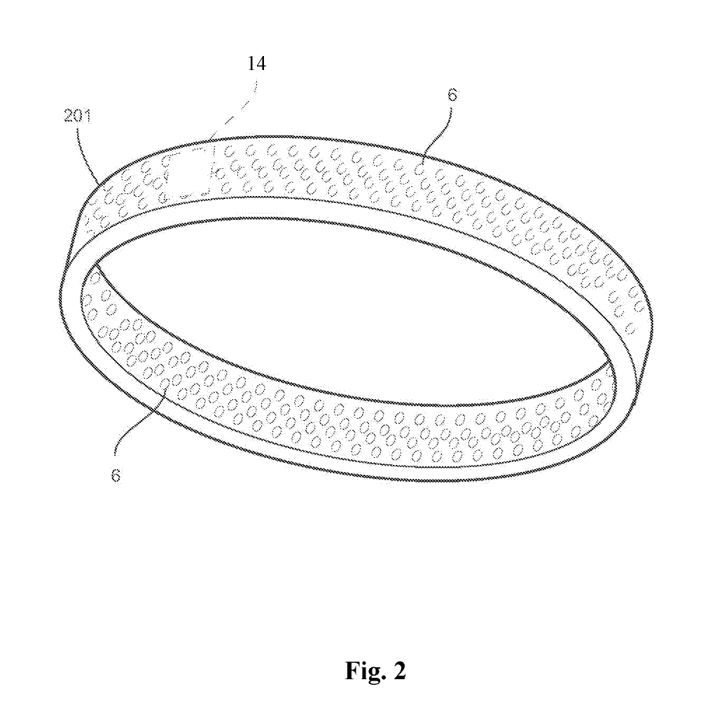

FIG. 2 shows a device in the form of a bracelet, which can functionally perform functions described for the smartwatch of FIG. 1.

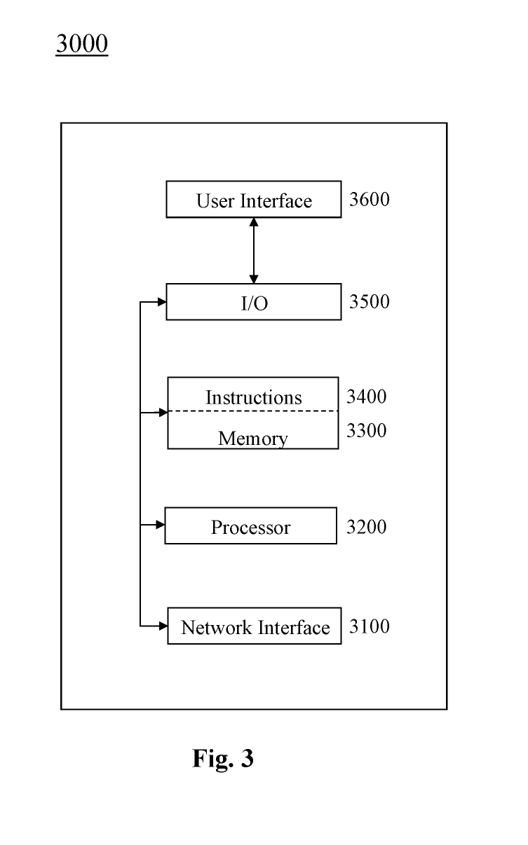

FIG. 3 is a block diagram of an exemplary embodiment of an information device 3000, which in certain operative embodiments can comprise, for example, electronic area 3 of FIG. 1 and/or peripheral devices coupled thereto. Information device 3000 can comprise any of numerous circuits and/or components, such as for example, one or more network interfaces 3100, one or more processors 3200, one or more memories 3300 containing instructions 3400, one or more input/output devices 3500, and/or one or more user interfaces 3600 coupled to one or more input/output devices 3500, etc.

In certain exemplary embodiments, via one or more user interfaces 3600, such as a graphical user interface, a user can view a rendering of information related to monitoring patient vital signs.

Certain exemplary embodiments can provide a relatively quick response to a coding patient in the form of a wireless bracelet or smartwatch used to notify medical personnel of patient vital signs and/or a deterioration thereof. Certain exemplary embodiments provide a substantially latex-free wristband, which can comprise an elastomer material and/or glass screen. Certain exemplary embodiments receive transmitted body temperature, respiratory rate, heart rate, blood pressure, and/or blood oxygenation level, etc. Certain exemplary embodiments provide a battery life of approximately 48 hours with a disinfecting charger port. Certain exemplary embodiments render information that can foreshadow cardiopulmonary arrests and/or other potential complications.

Certain exemplary embodiments can be designed primarily for the use of doctors and/or nurses. Certain exemplary embodiments can notify healthcare professionals when a patient's vital signs drop below or exceed a range of values that are personalized for each patient. Since the band is connected to a Wi-Fi network, it is synced to the nurses' computers along with the hospital monitors and is therefore able to detect changes in a patient's physical state immediately. Once one or more of these values drop below/rise above an individual's unique vital range, the particular patient's primary nurse is notified with a vibration and screen displaying the patient's room number, vital sign abbreviation, and an arrow indicating whether the value has dropped or risen. Once a patient experiences a life-threatening drop in the case of, for example, cardiac arrest, every nurse present on the unit can be notified when each of their bands vibrates and/or emits an alarming sound. Thus, the reaction of medical professionals can be reduced and a coding patient has a better chance of receiving help in a timely manner.

Certain exemplary embodiments can give nurses the ability to monitor multiple patients continuously. The alert system of such bracelets can decrease the average amount of time it takes for nurses and doctors to reach the room of a coding patient. Because this technology can notify nurses of when their patients vital signs fluctuate outside of the programmed normal range, the nurses can be more able to reach patients before their condition worsens and prevent further damage to patient health. In the future, as populations grow, more pressure and obligations will be placed on medical care facilities. In some circumstances, the amount of time that nurses have available for their patients' care can be limited. Certain exemplary embodiments can improve nurse efficiency and improve patient care.

In certain exemplary embodiments doctors and nurses can use this alert system to create a more personalized monitoring system. This is important because patients in different conditions have different warning signs for their condition worsening. Overall, the technology of this alert system can add to the more personalized hospitalization and care that the medical field has been advancing towards. As well as personalization, we hope that in coming years technology will advance enough so that other patient stats can be used in the alert system. Some of these measurement can comprise white blood cell count and/or red blood cell count, etc.

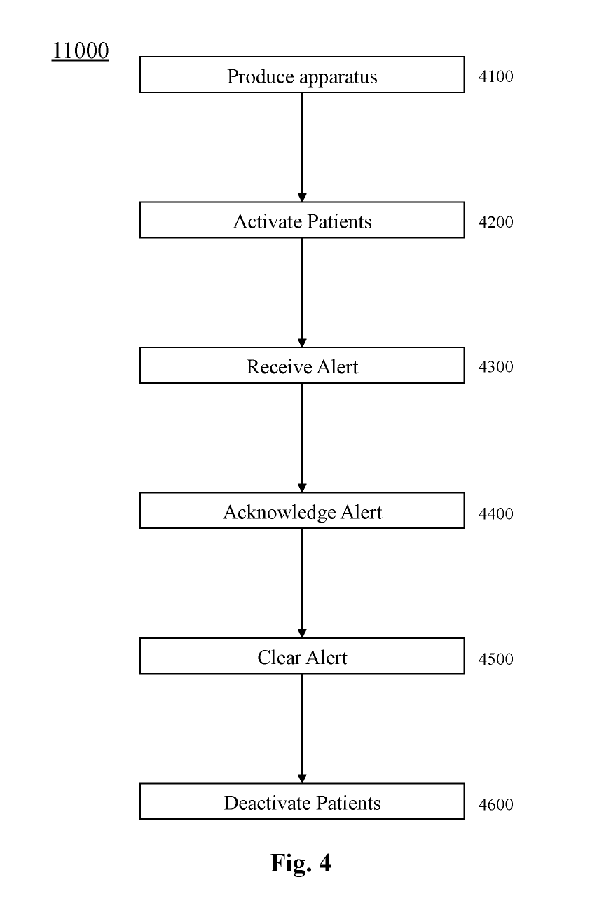

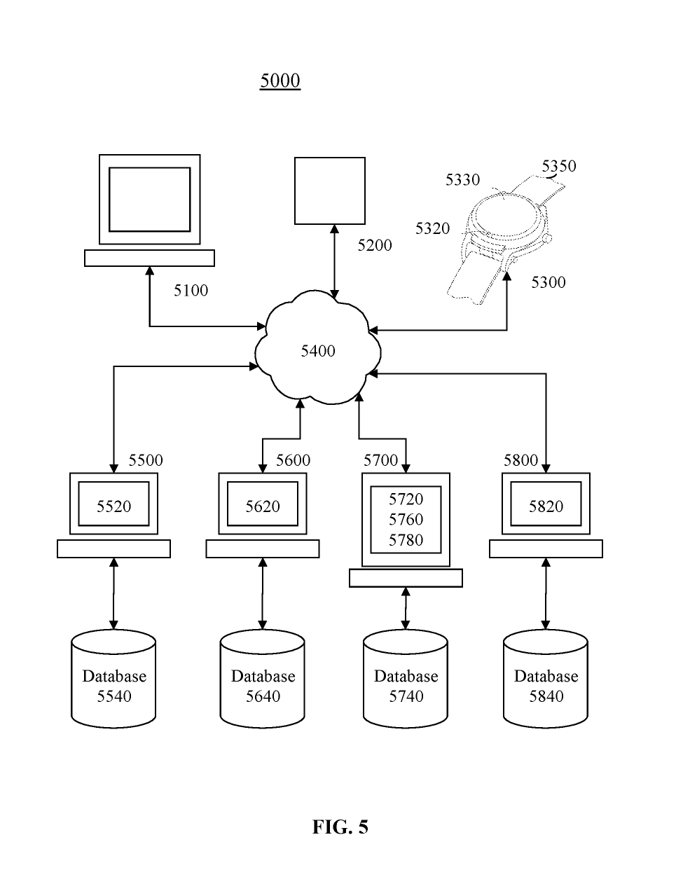

FIG. 4 is a flowchart of an exemplary embodiment of a method 4000. At activity 4100, a bracelet that receives patient vital sign information can be produced.

At activity 4200, the bracelet can be activated, such as via being communicatively coupled to a medical information system. At activity 4300, via information obtained from the medical information system, an alert can be received and a medical practitioner can be provided sensory information indicative of the alert.

At activity 4400, the medical practitioner can acknowledge and/or silence the alert.

At activity 4500, the alert can be cleared from the device such that the device is ready for a subsequent alert.

At activity 4600, the device can be deactivated. For example, the medical practitioner can deactivate the device at the end of a shift such that the device is no longer communicatively coupled to the medical information system.

FIG. 5 is a block diagram of an exemplary embodiment of a system 5000, which can comprise an apparatus 5300 (e.g., a bracelet, smartwatch, and/or smartphone, etc.), an information device 5100, tablet 5200, a network 5400 (e.g., a Wi-Fi network), a first server 5500, a second server 5600, a third server 5700, and a fourth server 5800. First server 5500 can comprise a first user interface 5520 and can be coupled to a first database 5540. Second server 5600 can comprise a second user interface 5620 and can be coupled to a second database 5640. Third server 5700 can comprise a third user interface 5720, a processor 5760, machine instructions 5780, and can be coupled to a third database 5740. Fourth server 5800 can comprise a fourth user interface 5820 and can be coupled to a fourth database 5840. Any of the methods and/or steps thereof can be carried out in whole or in part by tablet 5200, apparatus 5300, information device 5100 and/or first server 5500. Apparatus 5300 comprises a user interface 5330 and a button 5320. Button 5320 can be constructed to allow a user to select a particular patient of a plurality of patients for vital sign rendering. Second server 5600, third server 5700, and/or fourth server 5800 can each be associated with implementation of a system via which rides are provided to customers. In certain exemplary embodiments, system 5000 can be used to implement one or more methods disclosed herein.

Certain exemplary embodiments provide an apparatus 5300 wearable by a user on a wrist of the user, apparatus 5300 comprising:
a processor (e.g., processor 3200 of FIG. 3) communicatively coupled to an information device 5100 of a medical care facility, wherein information device 5100 is constructed to receive and store vital sign information for a plurality of patients of the medical care facility. The processor can be constructed to:
   responsive to a selection by the user, wirelessly request and receive values of one or more vital signs for a selected patient, the selected patient one of the plurality of patients;
   wirelessly receive information from information device 5100, the information comprising a variation of a vital sign of a patient from a predetermined threshold, the patient one of the plurality of patients; and/or
   cause an alarm responsive to the variation of a vital sign of the patient from the predetermined threshold; and/or
user interface 5330 constructed to render one or more of the values of one or more vital signs for a particular patient or information concerning the vital sign of the coding patient.

The predetermined threshold can be established for the patient indicative of cardiac failure and/or a significant detrimental change in the patient.

In certain exemplary embodiments, the predetermined threshold is a limit of a predetermined range for the vital sign. In certain exemplary embodiments, the predetermined threshold is a limit of a predetermined rate of change for the vital sign. Apparatus 5300 can comprise a wristband 5350 that is substantially latex free. Wristband 5350 can comprise an elastomer. User interface 5330 can comprise glass. Apparatus 5300 can vibrate and/or emit an audio alarm responsive to the variation of the vital sign of the coding patient from the predetermined threshold. The vital signs can comprise one or more of body temperature, respiratory rate, heart rate, blood pressure, blood oxygenation level, red blood cell count, and/or white blood cell count, etc. In certain exemplary embodiments, system 5000 can comprise one or more of:
information device 5100; and/or
a network 5400 (e.g., a Wi-Fi network) coupled to information device 5100 and apparatus 5300, wherein the vital sign information for the plurality of patients is transmitted to apparatus 5300 via network 5400.

### Definitions

When the following terms are used substantively herein, the accompanying definitions apply. These terms and definitions are presented without prejudice, and, consistent with the application, the right to redefine these terms during the prosecution of this application or any application claiming priority hereto is reserved. For the purpose of interpreting a claim of any patent that claims priority hereto, each definition (or redefined term if an original definition was amended during the prosecution of that patent), functions as a clear and unambiguous disavowal of the subject matter outside of that definition.
a—at least one.
account—to determine a cause.
accurate—substantially consistent with an actual value.
activate—to register with.
activity—an action, act, step, and/or process or portion thereof
adapted to—made suitable or fit for a specific use or situation.
adapter—a device used to effect operative compatibility between different parts of one or more pieces of an apparatus or system.
against—into contact with.
alarm—a signal perceptible to a human that serves to call attention to something.
algorithm—a set of rules followed in determinations.
analysis—determining one or more features of something via careful examination.
analyze—to examine carefully and in detail.
and/or—either in conjunction with or in alternative to.
apparatus—an appliance or device for a particular purpose.
arm—each of the two upper limbs of the human body from the shoulder to the hand.
associate—to join, connect together, and/or relate.
audible—perceptible by human hearing.
automatically—acting or operating in a manner essentially independent of external influence or control. For example, an automatic light switch can turn on upon “seeing” a person in its view, without the person manually operating the light switch.
average—to smooth a signal in a manner such that the signal magnitude approximates a mean of a plurality of signal values.
average population—a large number of people that in some sense are representative of a user.
backbone network—a “transit” network often made up of long-distance telephone trunk lines and/or other wired and/or wireless links such as microwave and satellite links for use in transmitting large amounts of data simultaneously between host computer systems connected to the Internet. Normal communicated data typically neither originates nor terminates in a backbone network.
bandpass filter—an electrical circuit constructed to attenuate substantially all frequencies except those of a specific band, which it amplifies.
base—a foundation for a determination.
baseline value—a standard magnitude of a parameter.
belong—to fit in a specified category or group.
between—with reference to a differentiation involving two or more things being considered together.
biometric—constructed to measure a physical characteristic.
biometric sensor—a sensor constructed to measure a physical characteristic. Exemplary biometric sensors comprise, for example, heart rate monitor, pulse transit time sensor, pulse oximeters, temperature sensors (e.g., thermometers), galvanometers, breathalyzers, carbon monoxide (CO) sensor, blood sugar, hygrometer, altimeter, barometer, GPS detector, accelerometer, gyroscope, magnetometer, EMG (electromyographic sensor), fingerprint reader, force transducer, audio sensor (e.g., microphone), proximity sensor, human velocity sensor, ambient light sensor, blood pressure sensor, flowmeters (e.g., laser Doppler flowmeters) or any combination thereof
blood oxygenation level—a quantity of oxygen saturated hemoglobin relative to total hemoglobin in human blood.
blood pressure—a force exerted by human blood on walls of blood vessels.
body temperature—a measure of kinetic energy of a human as measured via a thermometer or equivalent device.
boost—to increase.
bound—kept within limits.
bracelet—a band or circlet worn on a user's wrist.
brain wave—an electrical impulse in the brain.
button—a knob or icon on a piece of electrical or electronic equipment that is pressed to operate it.
calibrate—to correlate readings of an instrument with those of a standard in order to check the instrument's accuracy.
calibration profile for health parameters—a patterned set of correlated readings for a user for a particular physically measured value.
can—is capable of, in at least some embodiments.
cancel—to signal that an event will not take place.
capacitive—constructed to measure capacitance of a part of a human body.
capture—to obtain.
cardiac failure—a condition wherein a heart of a human is unable to pump sufficiently to maintain blood flow to meet needs of a body of the human.
case—a container constructed to hold something.
cause—to bring about.
certainty—the state of being statistically confident.
change—to become different.
charging mechanism—an electrical circuit constructed to provide electrical energy to a battery system.
circuit—an electrically conductive pathway and/or a communications connection established across two or more switching devices comprised by a network and between corresponding end systems connected to, but not comprised by the network.
coding—experiencing a medical emergency sufficient, according to normal standards of medical care, to warrant an alert to one or more staff personnel of a medical treatment facility to take substantially immediate action responsive to the alert.
communicate—to exchange information.
compare—to determine differences between two or more things.
compensate—to adjust for something.
compress—to apply a compression algorithm to electronic data so that the electronic data takes up less space when stored on a memory device.
comprising—including but not limited to.
concerning—pertaining to.
confidence interval—a statistical interval estimate of a population parameter.
confidence level—where confidence intervals are constructed across many separate data analyses of repeated experiments, the proportion of such intervals that contain the true value of a statistical parameter.
configure—to make suitable or fit for a specific use or situation.
connect—to join or fasten together.
constructed to—built for a specific use or situation.
constructed to—made to and/or designed to.
contact—to substantially directly touch.
convert—to transform, adapt, and/or change.
count—a total number of
couple—to link in some fashion.
coupleable—capable of being joined, connected, and/or linked together.
data—distinct pieces of information, usually formatted in a special or predetermined way and/or organized to express concepts.
data structure—an organization of a collection of data that allows the data to be manipulated effectively and/or a logical relationship among data elements that is designed to support specific data manipulation functions. A data structure can comprise meta data to describe the properties of the data structure. Examples of data structures can include: array, dictionary, graph, hash, heap, linked list, matrix, object, queue, ring, stack, tree, and/or vector.
database—an organized collection of data stored on a memory device.
decision tree—a tree algorithm in which the selection of each branch requires that some type of logical decision be made.
define—to establish the outline, form, or structure of
detect—to determine the existence of something.
determine—to obtain, calculate, decide, deduce, and/or ascertain.
deviation—the difference between one of a set of values and some fixed value.
device—a machine, manufacture, and/or collection thereof.
difference—a way in which things are not the same.
disturbance—a change in measured data of a physical system.
elastomer—a polymer with viscoelasticity (having both viscosity and elasticity) and very weak inter-molecular forces, generally having low Young's modulus and high failure strain compared with other materials.
electrocardiogram—a recording of electrical activity of a human heart.
electroencephalogram—a non-invasive method to record electrical activity of the brain along a scalp; EEG measures voltage fluctuations resulting from ionic current flows within neurons of a brain.
emergency medical system—a type of emergency service dedicated to providing medical care and/or transport to medical care.
energy—power derived from the utilization of physical or chemical resources.
entity—a person or organization that performs a function.
estimate—to calculate and/or determine approximately and/or tentatively.
extremity—a limb, such as a leg or arm or a part thereof (e.g., an ankle).
filter—a device that allows signals with certain properties, such as signals lying in a certain frequency range, to pass while blocking the passage of others.
finger—each of the four slender jointed parts attached to either hand (or five, if the thumb is included).
generate—to create, produce, give rise to, and/or bring into existence.
glass—a substantially transparent non-crystalline amorphous solid.
global positioning system—a space-based satellite navigation system that provides location and time information in all weather conditions, anywhere on or near the Earth where there is an unobstructed line of sight to four or more GPS satellites.
glucometer—a medical device constructed to determine an approximate concentration of glucose in the blood.
grip—to take a firm hold of.
haptic—involving the human sense of kinesthetic movement and/or the human sense of touch. Among the many potential haptic experiences are numerous sensations, body-positional differences in sensations, and time-based changes in sensations that are perceived at least partially in non-visual, non-audible, and non-olfactory manners, including the experiences of tactile touch (being touched), active touch, grasping, pressure, friction, traction, slip, stretch, force, torque, impact, puncture, vibration, motion, acceleration, jerk, pulse, orientation, limb position, gravity, texture, gap, recess, viscosity, pain, itch, moisture, temperature, thermal conductivity, and thermal capacity.
head—a portion of the human body located above the neck.
health parameters—a variable related to the physical well-being of a human.
heart condition—a medical issue with the cardiac muscle of a human.
heart rate—a frequency at which a heart beats over time.
heart rate monitor—a system constructed to measure a frequency of heartbeats.
heartbeat—a pulsation of the heart, including one substantially complete systole and diastole.
hydrophobic coating—a substantially water-proof covering.
indication—a degree marked by an instrument.
individually—one at a time.
information—knowledge gained through study, measurement, communication, research, and/or instruction, etc.
information device—any device capable of processing data and/or information, such as any general purpose and/or special purpose computer, such as a personal computer, workstation, server, minicomputer, mainframe, supercomputer, computer terminal, laptop, wearable computer, and/or Personal Digital Assistant (PDA), mobile terminal, Bluetooth device, communicator, “smart” phone (such as a Treo-like device), messaging service (e.g., Blackberry) receiver, pager, facsimile, cellular telephone, a traditional telephone, telephonic device, a programmed microprocessor or microcontroller and/or peripheral integrated circuit elements, an ASIC or other integrated circuit, a hardware electronic logic circuit such as a discrete element circuit, and/or a programmable logic device such as a PLD, PLA, FPGA, or PAL, or the like, etc. In general any device on which resides a finite state machine capable of implementing at least a portion of a method, structure, and/or or graphical user interface described herein may be used as an information device. An information device can comprise components such as one or more network interfaces, one or more processors, one or more memories containing instructions, and/or one or more input/output (I/O) devices, one or more user interfaces coupled to an I/O device, etc.
input/output (I/O) device—any sensory-oriented input and/or output device, such as an audio, visual, haptic, olfactory, and/or taste-oriented device, including, for example, a monitor, display, projector, overhead display, keyboard, keypad, mouse, trackball, joystick, gamepad, wheel, touchpad, touch panel, pointing device, microphone, speaker, video camera, camera, scanner, printer, haptic device, vibrator, tactile simulator, and/or tactile pad, potentially including a port to which an I/O device can be attached or connected.
install—to connect or set in position and prepare for use.
insufficient—lacking something that is needed.
intense physical activity—something that a human does that increases a heart rate by over fifty percent above a resting heart rate.
laser Doppler flowmeter—a device that uses a Doppler shift in a laser beam to measure a fluid flow velocity.
latex free—substantially lacking a substance derived from a milky fluid found in 10% of all flowering plants (angiosperms), wherein the milky fluid comprises proteins, alkaloids, starches, sugars, oils, tannins, resins, and gums that coagulate on exposure to air.
lead—an electrical connection such as a length of wire or metal pad that comes from a device.
limit—a largest or smallest value of a vital sign that is not indicative of a patient coding.
location—a particular position.
machine instructions—directions adapted to cause a machine, such as an information device, to perform one or more particular activities, operations, or functions. The directions, which can sometimes form an entity called a “processor”, “kernel”, “operating system”, “program”, “application”, “utility”, “subroutine”, “script”, “macro”, “file”, “project”, “module”, “library”, “class”, and/or “object”, etc., can be embodied as machine code, source code, object code, compiled code, assembled code, interpretable code, and/or executable code, etc., in hardware, firmware, and/or software.
machine readable medium—a physical structure from which a machine can obtain data and/or information. Examples include a memory, punch cards, etc.
may—is allowed and/or permitted to, in at least some embodiments.
measure—to ascertain an extent of.
measurement—an extent of a variable.
medical care facility—a location constructed to provide care to humans experiencing health concerns and/or problems.
medical information—data concerning the physical health of a human.
memory device—an apparatus capable of storing analog or digital information, such as instructions and/or data. Examples include a non-volatile memory, volatile memory, Random Access Memory, RAM, Read Only Memory, ROM, flash memory, magnetic media, a hard disk, a floppy disk, a magnetic tape, an optical media, an optical disk, a compact disk, a CD, a digital versatile disk, a DVD, and/or a raid array, etc. The memory device can be coupled to a processor and/or can store instructions adapted to be executed by processor, such as according to an embodiment disclosed herein.
method—a process, procedure, and/or collection of related activities for accomplishing something.
model—a standard that can be used for a comparison.
motion—a bodily movement.
movement—an act of changing position.
network—a communicatively coupled plurality of nodes. A network can be and/or utilize any of a wide variety of sub-networks, such as a circuit switched, public-switched, packet switched, data, telephone, telecommunications, video distribution, cable, terrestrial, broadcast, satellite, broadband, corporate, global, national, regional, wide area, backbone, packet-switched TCP/IP, Fast Ethernet, Token Ring, public Internet, private, ATM, multi-domain, and/or multi-zone sub-network, one or more Internet service providers, and/or one or more information devices, such as a switch, router, and/or gateway not directly connected to a local area network, etc.
network interface—any device, system, or subsystem capable of coupling an information device to a network. For example, a network interface can be a telephone, cellular phone, cellular modem, telephone data modem, fax modem, wireless transceiver, Ethernet card, cable modem, digital subscriber line interface, bridge, hub, router, or other similar device.
non-medical device mode—a user selectable functional state of a system that is constructed to provide medical information but not a medical diagnosis and/or medical advice.
notify—to give notice or report something.
obtain—to acquire.
P wave—an electrocardiogram pattern representing atrial depolarization, which results in atrial contraction.
packet—a discrete instance of communication.
partially—substantially less than totally.
particular—a selected thing from a plurality of things.
patient—a human being evaluated and/or treated at a medical care facility.
Peak-Peak interval—an elapsed time between successive peaks of a signal.
photoplethysmogram—a volumetric measurement of a human organ.
photoplethysmogram—an optically obtained volumetric measurement of an organ.
plurality—the state of being plural and/or more than one.
population norm—a standard value for a large number of people.
power supply—an energy source for a device and/or system.
PR interval—the period, measured in milliseconds, that extends from the beginning of the P wave (the onset of atrial depolarization) until the beginning of the QRS complex (the onset of ventricular depolarization); it is normally between 120 and 200 ms in duration.
predetermined—established in advance.
predict—to determine in advance.
press—to apply pressure to.
prior—preceding in time.
processor—a device and/or set of machine-readable instructions for performing one or more predetermined tasks. A processor can comprise any one or a combination of hardware, firmware, and/or software. A processor can utilize mechanical, pneumatic, hydraulic, electrical, magnetic, optical, informational, chemical, and/or biological principles, signals, and/or inputs to perform the task(s). In certain embodiments, a processor can act upon information by manipulating, analyzing, modifying, converting, transmitting the information for use by an executable procedure and/or an information device, and/or routing the information to an output device. A processor can function as a central processing unit, local controller, remote controller, parallel controller, and/or distributed controller, etc. Unless stated otherwise, the processor can be a general-purpose device, such as a microcontroller and/or a microprocessor, such the Pentium IV series of microprocessor manufactured by the Intel Corporation of Santa Clara, Calif. In certain embodiments, the processor can be dedicated purpose device, such as an Application Specific Integrated Circuit (ASIC) or a Field Programmable Gate Array (FPGA) that has been designed to implement in its hardware and/or firmware at least a part of an embodiment disclosed herein.
profile information—data concerning a user.
project—to calculate, estimate, or predict.
prompt—a message or symbol on a user interface requesting input from a user.
provide—to furnish, supply, give, and/or make available.
pulse oximeter—an instrument constructed to measure oxygen saturation of hemoglobin in a sample of blood.
pulse wave—a kind of non-sinusoidal waveform that is similar to a square wave, but does not have a symmetrical shape associated with a perfect square wave.
QRS complex—the combination of three of the graphical deflections seen on an electrocardiogram.
QT interval—a measure of the time between the start of the Q wave and the end of the T wave in the heart's electrical cycle.
rate of change—an extent to which a measured variable changes over a predetermined time interval.
reading—a signal indicative of a measured physical value.
receive—to get as a signal, take, acquire, and/or obtain.
recommend—to suggest, praise, commend, and/or endorse.
reconstruct—to form a model of from measured data.
relay—to retransmit a signal.
remote—far apart; separable by a distance of at least one mile.
render—to make perceptible to a human, for example as data, commands, text, graphics, audio, video, animation, and/or hyperlinks, etc., such as via any visual, audio, and/or haptic means, such as via a display, monitor, electric paper, ocular implant, cochlear implant, speaker, etc.
repeatedly—again and again; repetitively.
request—to express a desire for and/or ask for.
respiratory rate—a frequency at which a human inhales and exhales.
responsive—acting in reaction to a stimulus.
run—to move swiftly on foot.
select—to choose from a plurality of choices.
selective—characterized by careful choice.
sensor—a device that detects events or changes in quantities and provides a corresponding output, generally as an electrical or optical signal.
set—a related plurality.
signal—information, such as machine instructions for activities and/or one or more letters, words, characters, symbols, signal flags, visual displays, and/or special sounds, etc. having prearranged meaning, encoded as automatically detectable variations in a physical variable, such as a pneumatic, hydraulic, acoustic, fluidic, mechanical, electrical, magnetic, optical, chemical, and/or biological variable, such as power, energy, pressure, flowrate, viscosity, density, torque, impact, force, frequency, phase, voltage, current, resistance, magnetomotive force, magnetic field intensity, magnetic field flux, magnetic flux density, reluctance, permeability, index of refraction, optical wavelength, polarization, reflectance, transmittance, phase shift, concentration, and/or temperature, etc. Depending on the context, a signal and/or the information encoded therein can be synchronous, asynchronous, hard real-time, soft real-time, non-real time, continuously generated, continuously varying, analog, discretely generated, discretely varying, quantized, digital, broadcast, multicast, unicast, transmitted, conveyed, received, continuously measured, discretely measured, processed, encoded, encrypted, multiplexed, modulated, spread, de-spread, demodulated, detected, de-multiplexed, decrypted, and/or decoded, etc.
significant detrimental change—a determined difference in measured physiological values that have been determined to have an important negative consequence in human physiology.
smart blood pressure sensor—a device and/or system that measures blood pressure via a plurality of sensors.
smartwatch—a device that comprises a band that is constructed to be worn on a user's wrist and has an ability to render information, the information comprising time and a vital sign of one or more patients.
snapshot—a measured physiological data set obtained over a predetermined time period.
ST segment depression—a value determined by measuring the vertical distance between the patient's trace and the isoelectric line at a location approximately 2-3 millimeters from the QRS complex.
ST segment elevation—an indicator of a myocardial infarction (i.e., heart attack) determined to have occurred via a measurement of a higher than normal connection between the QRS complex and the T wave in an electrocardiogram.
standard—an object or value that bears a defined relationship to a unit of measure used for calibration of measuring devices.
store—to place, hold, and/or retain data, typically in a memory.
stroke—the loss of brain function due to a disturbance in the blood supply to the brain.
submit—to present to an information device.
substantially—to a great extent or degree.
support—to bear the weight of, especially from below.
surround—to be substantially all around something.
system—a collection of mechanisms, devices, machines, articles of manufacture, processes, data, and/or instructions, the collection designed to perform one or more specific functions.
temporal region—the side of a human head posterior to the eyes.
threshold—a point that when exceeded produces a given effect or result.
time—a quantity measuring duration, usually with reference to a periodic process such as the rotation of the earth or the vibration of electromagnetic radiation emitted from certain atoms.
time period—an interval of time.
touch—to contact.
transmission—a conveyance of data from one location to another.
transmit—to send as a signal, provide, furnish, and/or supply.
transmitter/receiver—a system constructed to send and acquire signals.
user—a person wearing, and being monitored by, an apparatus.
user interface—any device for rendering information to a user and/or requesting information from the user. A user interface includes at least one of textual, graphical, audio, video, animation, and/or haptic elements. A textual element can be provided, for example, by a printer, monitor, display, projector, etc. A graphical element can be provided, for example, via a monitor, display, projector, and/or visual indication device, such as a light, flag, beacon, etc. An audio element can be provided, for example, via a speaker, microphone, and/or other sound generating and/or receiving device. A video element or animation element can be provided, for example, via a monitor, display, projector, and/or other visual device. A haptic element can be provided, for example, via a very low frequency speaker, vibrator, tactile stimulator, tactile pad, simulator, keyboard, keypad, mouse, trackball, joystick, gamepad, wheel, touchpad, touch panel, pointing device, and/or other haptic device, etc. A user interface can include one or more textual elements such as, for example, one or more letters, number, symbols, etc. A user interface can include one or more graphical elements such as, for example, an image, photograph, drawing, icon, window, title bar, panel, sheet, tab, drawer, matrix, table, form, calendar, outline view, frame, dialog box, static text, text box, list, pick list, pop-up list, pull-down list, menu, tool bar, dock, check box, radio, hyperlink, browser, button, control, palette, preview panel, color wheel, dial, slider, scroll bar, cursor, status bar, stepper, and/or progress indicator, etc. A textual and/or graphical element can be used for selecting, programming, adjusting, changing, specifying, etc. an appearance, background color, background style, border style, border thickness, foreground color, font, font style, font size, alignment, line spacing, indent, maximum data length, validation, query, cursor type, pointer type, autosizing, position, and/or dimension, etc. A user interface can include one or more audio elements such as, for example, a volume control, pitch control, speed control, voice selector, and/or one or more elements for controlling audio play, speed, pause, fast forward, reverse, etc. A user interface can include one or more video elements such as, for example, elements controlling video play, speed, pause, fast forward, reverse, zoom-in, zoom-out, rotate, and/or tilt, etc. A user interface can include one or more animation elements such as, for example, elements controlling animation play, pause, fast forward, reverse, zoom-in, zoom-out, rotate, tilt, color, intensity, speed, frequency, appearance, etc. A user interface can include one or more haptic elements such as, for example, elements utilizing tactile stimulus, force, pressure, vibration, motion, displacement, temperature, etc.
variation—a state and/or degree of difference.
velocity—a time rate of change of position of a body in a specified direction.
via—by way of and/or utilizing.
vibrate—to oscillation about an equilibrium point.
vital sign—one of a group of a plurality of measureable indicators of the status of a human body's life-sustaining functions. These indicators can be used to assess the general physical health of a human, give clues to possible diseases, and show progress toward recovery. Normal ranges for a human's vital signs can vary with age, weight, gender, and overall health. A vital sign can be body temperature, blood pressure, pulse (i.e., heart rate), and/or breathing rate (i.e., respiratory rate), etc.
warning—information that alerts someone of a potentially harmful condition.
wear—to have on one's body or a part of one's body.
Wi-Fi—a technology for wireless local area networking with devices based on IEEE 802.11 standards.
wireless—using radio, microwaves, etc. (as opposed to wires or cables) to transmit signals.
wrist—a joint connecting the hand with the forearm of a human.
wristband—a strap worn around a wrist of a human.

### Note

Still other substantially and specifically practical and useful embodiments will become readily apparent to those skilled in this art from reading the above-recited and/or herein-included detailed description and/or drawings of certain exemplary embodiments. It should be understood that numerous variations, modifications, and additional embodiments are possible, and accordingly, all such variations, modifications, and embodiments are to be regarded as being within the scope of this application.

Thus, regardless of the content of any portion (e.g., title, field, background, summary, description, abstract, drawing figure, etc.) of this application, unless clearly specified to the contrary, such as via explicit definition, assertion, or argument, with respect to any claim, whether of this application and/or any claim of any application claiming priority hereto, and whether originally presented or otherwise:
there is no requirement for the inclusion of any particular described or illustrated characteristic, function, activity, or element, any particular sequence of activities, or any particular interrelationship of elements;
no characteristic, function, activity, or element is “essential”;
any elements can be integrated, segregated, and/or duplicated;
any activity can be repeated, any activity can be performed by multiple entities, and/or any activity can be performed in multiple jurisdictions; and
any activity or element can be specifically excluded, the sequence of activities can vary, and/or the interrelationship of elements can vary.

Moreover, when any number or range is described herein, unless clearly stated otherwise, that number or range is approximate. When any range is described herein, unless clearly stated otherwise, that range includes all values therein and all subranges therein. For example, if a range of 1 to 10 is described, that range includes all values therebetween, such as for example, 1.1, 2.5, 3.335, 5, 6.179, 8.9999, etc., and includes all subranges therebetween, such as for example, 1 to 3.65, 2.8 to 8.14, 1.93 to 9, etc.

When any claim element is followed by a drawing element number, that drawing element number is exemplary and non-limiting on claim scope. No claim of this application is intended to invoke paragraph six of 35 USC 112 unless the precise phrase “means for” is followed by a gerund.

Any information in any material (e.g., a United States patent, United States patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such material is specifically not incorporated by reference herein.

Accordingly, every portion (e.g., title, field, background, summary, description, abstract, drawing figure, etc.) of this application, other than the claims themselves, is to be regarded as illustrative in nature, and not as restrictive, and the scope of subject matter protected by any patent that issues based on this application is defined only by the claims of that patent.

What is claimed is:

1. A system comprising:
an bracelet wearable by a user on a wrist of the user, the bracelet comprising:
a processor communicatively coupled to an information device of a medical care facility, wherein the information device is constructed to receive and store vital sign information for a plurality of patients of the medical care facility, the processor constructed to:
wirelessly receive information from the information device, the information comprising a variation of a vital sign of a patient from a predetermined threshold established for the patient, the variation of the vital sign indicative of a cardiac failure, the patient one of the plurality of patients; and
cause an alarm responsive to the cardiac failure.

2. The of claim 1, wherein:
the processor is constructed to, responsive to a selection by the user, wirelessly request and receive values of one or more vital signs for a selected patient, the selected patient one of the plurality of patients; and
the bracelet comprises a user interface constructed to render the received values for the one or more vital signs.

3. The of claim 1, wherein:
the bracelet comprises a user interface constructed to render information concerning the cardiac failure.

4. The of claim 1, wherein:
the alarm is a vibration.

5. The of claim 1, wherein:
the alarm is audible.

6. The of claim 1, wherein:
the predetermined threshold is a limit of a predetermined range for the vital sign.

7. The of claim 1, wherein:
the predetermined threshold is a limit of a predetermined rate of change for the vital sign.

8. The of claim 1, wherein:
the bracelet comprises a wristband that is substantially latex free, the wristband comprising an elastomer.

9. The of claim 1, wherein:
the bracelet comprises a user interface that comprises glass.

10. The of claim 1, wherein:
the bracelet vibrates responsive to the variation of the vital sign of the coding patient from the predetermined threshold.

11. The of claim 1, wherein:
the vital signs comprise one or more of body temperature, respiratory rate, heart rate, blood pressure, and blood oxygenation level.

12. The system of claim 1, further comprising:
the information device.

13. The system of claim 1, further comprising:
a Wi-Fi network coupled to the information device and the bracelet, wherein the vital sign information for the plurality of patients is transmitted to the bracelet via the Wi-Fi network.

14. A system comprising:
an bracelet wearable by a user on a wrist of the user, the bracelet comprising:
a processor communicatively coupled to an information device of a medical care facility, wherein the information device is constructed to receive and store vital sign information for a plurality of patients of the medical care facility, the processor constructed to:
wirelessly receive information from the information device, the information comprising a variation of a vital sign of a patient from a predetermined threshold established for the patient, the variation of the vital sign indicative of a significant detrimental change in the patient, the patient one of the plurality of patients; and
cause an alarm responsive to the significant detrimental change.
